(19) United States
(12) Patent Application Publication
(10) Pub. No.: US 2025/0360008 A1
(43) Pub. Date: Nov. 27, 2025

(54) Device with Deformable Spheres

(71) Applicant: President and Fellows of Harvard College, Cambridge, MA (US)

(72) Inventor: Abderrahmane Adel Tayeb Djellouli, Cambridge, MA (US)

(21) Appl. No.: 19/196,280

(22) Filed: May 1, 2025

Related U.S. Application Data
(60) Provisional application No. 63/651,350, filed on May 23, 2024.
(60) Provisional application No. 63/690,398, filed on Sep. 4, 2024.

(51) Int. Cl. A61F 2/94

(52) U.S. Cl. CPC A61F 2/94; A61F 2230/0006; A61F 2250/0013

(57) ABSTRACT

A device includes a tube extending between a first end and a second end. The tube forms a rigid structure having an internal surface that defines a hollow interior area of the tube. One or more compressible structures are seeded on and extend into the hollow interior of the tube from the internal surface. The one or more compressible structures allow a flow path through the hollow interior area.

### STATEMENT REGARDING FEDERALLY SPONSORED RESEARCH

This invention was made with government support under 2011754 awarded by National Science Foundation (NSF). The government has certain rights in this invention.

### FIELD OF THE INVENTION

The present invention relates generally to a tubular device having deformable spheres arranged along an internal surface of the device, and in particular to a tubular device having deformable spheres arranged along an internal surface of the device that collapse to maintain a threshold pressure for fluid flowing through the device.

### BACKGROUND OF THE INVENTION

The pursuit of materials having enhanced functionality has led to the emergence of artificially engineered materials whose properties are determined by structure rather than by composition. Such artificially engineered materials are commonly referred to as metamaterials. Through careful design of their building blocks, metamaterials with unprecedented mechanical properties have been realized. Metamaterials have the potential to revolutionize medical devices including, for example without limitation, arterial implants.

Traditional arterial implants do not include a mechanism for limiting blood pressure through the artery. A need exists for a device having a response to pressure that can be tailored to limit the pressure of the fluid flowing through the device. The present disclosure provides a solution to these and other needs.

### SUMMARY OF THE INVENTION

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

According to certain aspects of the present disclosure, a device includes a tube extending between a first end and a second end. The tube forms a rigid structure having an internal surface that defines a hollow interior area of the tube. One or more compressible structures seeded on and extending into the hollow interior of the tube from the internal surface. The one or more compressible structures allowing a flow path through the hollow interior area.

According to some features of the above aspects, each of the one or more compressible structures has an external shell defining an internal volume between the external shell and the internal surface of the tube. The external shell is deformable between a first original shape and a second deformed shape in response to a pressure increase in the hollow interior of the tube.

According to some features of the above aspects, the external shell is deformable based on a pressure change between a first threshold pressure and a second threshold pressure.

According to some features of the above aspects, the second deformed shape is one of a plurality of second deformed shapes.

According to some features of the above aspects, the internal volume of the second deformed shape decreases as pressure is increased in the hollow interior of the tube.

According to some features of the above aspects, the external shell of the one or more compressible structures has a surface that is at least partially spherical.

According to some features of the above aspects, the internal volume is a vacuum.

According to some features of the above aspects, the first threshold pressure is determined by material properties of the external shell and a ratio of the thickness to the radius of the external shell.

According to some features of the above aspects, a fluid is fully enclosed within the external shell.

According to some features of the above aspects, the fluid is air.

According to some features of the above aspects, the first threshold pressure is determined by material properties of the external shell, an internal pressure of the fluid within the external shell, and a ratio of the thickness to the radius of the external shell.

According to some features of the above aspects, the tube is in the form of a stent that is adapted for placement in a blood vessel.

According to some features of the above aspects, the first threshold pressure is adapted to be a maximum healthy blood pressure.

According to some features of the above aspects, the flow path is configured to receive a fluid.

According to some features of the above aspects, the fluid is a liquid.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure, and its advantages and drawings, will be better understood from the following description of representative embodiments together with reference to the accompanying drawings. These drawings depict only representative embodiments and are therefore not to be considered as limitations on the scope of the various embodiments or claims.

FIG. 1 shows a schematic view of device having internal compressible structures viewed through an end of the device, according to certain aspects of the present disclosure.

FIG. 2 shows a cross-sectional schematic view taken along the lines 2-2 of the device of FIG. 1, according to certain aspects of the present disclosure.

FIG. 3 shows a schematic view of compressible structures seeded on an internal surface of a tube, the compressible structures having a first shape, according to certain aspects of the present disclosure.

FIG. 4 shows a schematic view of compressible structures seeded on an internal surface of a tube, the compressible structures having a second shape, according to certain aspects of the present disclosure.

FIG. 5 shows a schematic view of compressible structures seeded on an internal surface of a tube, the compressible structures having a second shape different from the second shape shown in FIG. 4, according to certain aspects of the present disclosure.

FIG. 6 shows a cross-sectional schematic representation of an exemplary compressible structure seeded on the internal wall of a tube, according to certain aspects of the present disclosure.

FIG. 7 shows an exemplary graph of pressure vs. time for a stent having internal compressible structures and implanted in an artery, according to certain aspects of the present disclosure.

### DETAILED DESCRIPTION

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

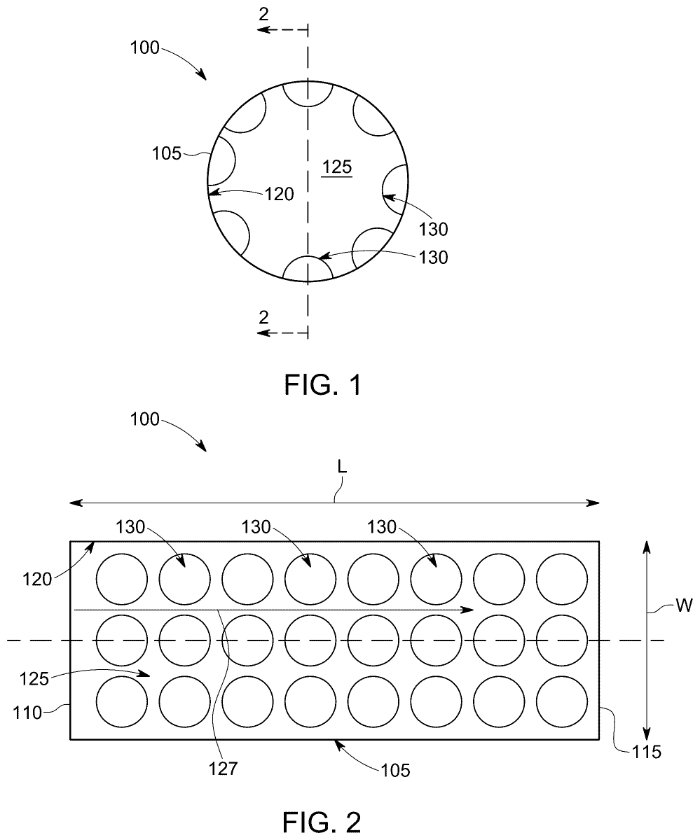

Referring generally to FIGS. 1 and 2, in an embodiment a device 100 includes a tube 105 extending between a first end 110 and a second end 115. In an embodiment, the tube 105 forms a rigid structure having an internal surface 120 that defines a hollow interior 125 of the tube 105. In other embodiments the tube 105 forms a flexible, conformable, or elastic structure. In an embodiment, one or more compressible structures 130 are seeded on the internal surface 120. The one or more compressible structures 130 extend into the hollow interior 125 of the tube 105 from the internal surface 120. The one or more compressible structures 130 allow a flow path 127 through the hollow interior 125. The flow path 127 through the hollow interior 125 is configured to receive a fluid. In an embodiment the fluid is a liquid. In another embodiment the fluid is a mixture of a liquid and particles suspended within the liquid, for example, blood.

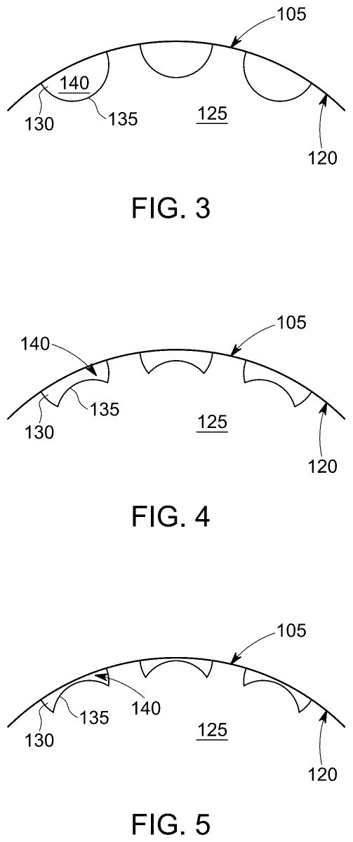

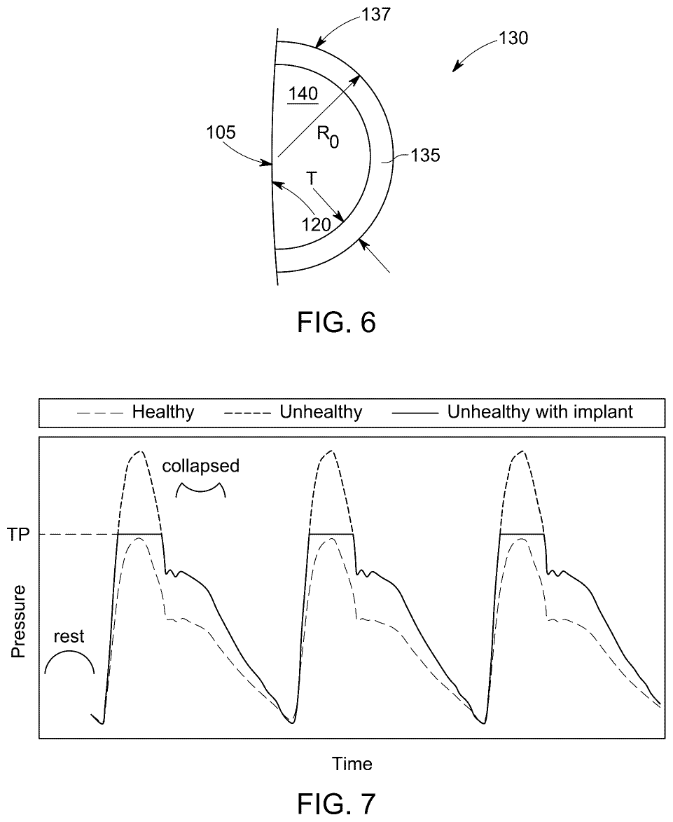

Referring generally to FIGS. 3-5, each of the one or more compressible structures 130 has an external shell 135 defining a hollow internal volume 140 between the external shell 135 and the internal surface 120 of the tube 105. In an embodiment, a surface 137 (see FIG. 6) of the external shell 135 of each of the compressible structures 130 is at least partially spherical, so that the shape of each of the external shells 135 is at least partially spheroidal. In other embodiments, the external shell 135 of the compressible structures 130 can have other shapes, including, for example without limitation, ovoid, ellipsoid, or combinations thereof. In an embodiment, a fluid, for example a liquid or gas, is fully enclosed within the internal volume 140 of the external shell 135 of the compressible structures 130. In an embodiment the gas is air. In an embodiment the internal volume 140 of the external shell 135 is a vacuum.

The external shell 135 is deformable between a first original shape, for example as shown in FIG. 3, and a second deformed shape, for example as shown in FIG. 4, in response to a pressure increase in the hollow interior 125 of the tube 105. As noted above, the first original shape can be spheroid, ovoid, ellipsoid, or combinations thereof. FIGS. 4 and 5 respectively illustrate two of a plurality of second deformed shapes. The internal volume 140 of the second deformed shape decreases (as shown going from FIG. 4 to FIG. 5) as pressure is increased in the hollow interior 125 of the tube 105.

Referring now to FIG. 6, each compressible structure 130 has the external shell 135 having the external surface 137. For a compressible structure 130 having a spheroidal shape, for example as shown in FIG. 6, the external surface 137 has a radius indicated by R₀, and the external shell 135 has a wall thickness indicated by T. For example, according to one illustrative example, the radius R₀ is about 2.5 millimeters (“mm”) and the thickness T is about 0.5 mm. The radius Ro and the wall thickness T can be sized as desired for particular operating conditions or a particular application.

Referring again generally to FIGS. 3-5, in an embodiment, the external shell 135 is deformable based on a pressure change between a first threshold pressure and a second threshold pressure. The deformation is an elastic deformation that can be reversed and repeated as needed. In an embodiment, the pressure at which the external shell 135 elastically deforms from the first shape (e.g., as shown in FIG. 3) to the second shape (e.g., as shown in FIG. 4), is different from the pressure at which the external shell 135 elastically deforms from the second shape (e.g., as shown in FIG. 4) back to the first shape example (e.g., as shown in FIG. 3).

For example, a first elastic deformation of the external shell 135, such as the change in shape between FIGS. 3 and 4, is the result of increasing the pressure on the external shell 135 from (i) a pressure that is less than a critical buckling pressure of the external shell 135 to (ii) the critical buckling pressure of the external shell 135. As the pressure in the hollow interior 125 of the tube 105 rises, the external shell 135 of the one or more compressible structures 130 may compress but will initially maintain the first shape, which in this example is spherical. However, when the pressure in the hollow interior 125 has risen sufficiently, the external shell 135 buckles and elastically deforms from its first shape to a second shape. This buckling of the external shell 135 decreases the internal volume 140 of the external shell 135. The buckling of the external shell 135 is therefore accompanied by an instantaneous small decrease in the pressure in the hollow interior 125 due to the instantaneous small increase in the volume of the hollow interior 125. The pressure at which the external shell 135 buckles is called the critical buckling pressure.

In an embodiment, the external shell 135 is further elastically deformable under pressure to withstand a second elastic deformation from the second shape (e.g., as shown in FIG. 4) back to the first shape (e.g., as shown in FIG. 3). The second elastic deformation of the external shell 135 is the result of decreasing the pressure on the external shell 135 from (i) a pressure that is greater than a critical expansion pressure of the external shell 135 to (ii) the critical expansion pressure of the external shell 135. As the pressure in the hollow interior 125 of the tube 105 falls, the external shell 135 of the one or more compressible structures 130 may expand but will initially remain buckled, having the characteristic dimple shape. However, when the pressure in the hollow interior 125 has fallen sufficiently, the external shell 135 expands and elastically deforms from a second shape back to the first shape. This expansion of the external shell 135 increases the internal volume 140 of the external shell 135. The expansion of the external shell 135 is therefore accompanied by an instantaneous small increase in the pressure in the hollow interior 125 due to the instantaneous small decrease in the volume of the hollow interior 125. The pressure at which the external shell 135 expands is called the critical expansion pressure.

Including a plurality of compressible structures 130 within the tube 105 has the effect of limiting the pressure of the hollow interior 125, or of any fluid flowing through the hollow interior 125, to the critical expansion pressure of the external shells 135. This result is true because the buckling event for each of a plurality external shells 135 occurs at about the critical buckling pressure.

For an example of how a plurality of compressible structures 130 would likely function to limit the pressure in the hollow interior 125, consider the following scenario where pressure in fluid flowing through the hollow interior 125 was rising due to some external influence, for example a pumping force on the fluid. Each time the external shell 135 of a compressible structure 130 buckled, there would be a small instantaneous drop in pressure, but the external influence in this example, the pumping force, would force the pressure back up to the critical buckling pressure. If there was only one compressible structure 130, the pressure supplied by the pumping force could continue to rise after the first external shell 135 buckled.

However, if there were a plurality of compressible structures 130, a steadily increasing pressure in the hollow interior 125 would trigger a second buckling event for the second external shell 135, followed by a third buckling event the third external shell 135, and so forth. The steadily increasing pressure in the hollow interior 125 would continue to trigger buckling of each subsequent external shell 135, until all of the pressure as supplied by the pumping force in excess of the critical buckling pressure was accounted for or used up by buckling the external shells 135. The net effect would be that the pressure of the fluid flowing through the hollow interior 125 would be limited to the critical buckling pressure of the external shells 135 of the compressible structures 130.

According to an exemplary embodiment, the critical expansion pressure is less than the critical buckling pressure. This is likely due to the tendency of a curved three-dimensional structure to resist buckling while also tending to return from a deformed buckled shape back to a non-deformed shape. In practice, this means that the one or more compressible structures 130, once having buckled at the critical buckling pressure will remain in the second shape (buckled) as the pressure is reduced below the critical buckling pressure until the pressure reaches the critical expansion pressure.

The critical buckling pressure is determined by material properties of the external shell 135, for example a bulk modulus or shear modulus of the material, an internal pressure of any fluid, including any liquid or gas, that may be in the internal volume 140 of the external shell 135, and a ratio of the thickness, T, to the radius, R₀, of the external shell 135. It has been observed that if a structure volume fraction φ is defined as a sum of the internal volumes 140 of the compressible structures 130 (each having the first shape) divided by a total volume of the tube 105 as defined by the internal surface 120, the critical buckling pressure is independent of the structure volume fraction φ. Thus, the critical buckling pressure can be tailored by selecting the size, shape, material, thickness, fill gas, and pressure of the fill gas of the compressible structures. This also means that the critical buckling pressure for a tube 105 having a single compressible structure 130 is the same as the critical buckling pressure for a tube 105 having a plurality of compressible structures 130. As noted above, a tube 105 having multiple compressible structures 130 allows for greater control of pressure, for example, in a more severely varying pressure environment.

In embodiment, the tube 105 is in the form of a stent 105 that is adapted for placement in a blood vessel. In this embodiment, the fluid flowing through the hollow interior 125 of the tube 105 is blood. In the context of the example of an external influence on the fluid as discussed above, the pumping force supplied to the fluid is a patient's heart. Referring to FIG. 7, a plot of pressure of the blood flowing through the stent 105 versus time is shown for three conditions. In a first condition, labeled as “healthy” and indicated by the long dashed lines, the blood pressure never exceeds a threshold pressure (labeled as TP). In a second condition, labeled as “unhealthy” and indicated by the short dashed lines, the blood pressure cyclically exceeds the threshold pressure. In a third condition, labeled as “unhealthy with implant” and indicated by the solid lines, the pressure of the blood flowing through the tube 105 is capped at the threshold pressure TP, which in this example, is the maximum healthy blood pressure for the patient.

Still referring to FIG. 7, the threshold pressure TP corresponds to the critical buckling pressure for the exterior shell 135 of the one or more compressible structures 130 that are seeded along the internal surface 120 of the tube 105. In an embodiment, the tube 105 has a length, L, (see FIG. 2) of about 10 to 50 mm. In an embodiment, the tube 105 has a diameter, W, (see FIG. 2) of about 10 to 20 mm. In other embodiments the tube 105 can have length, L, and width, W, dimensions that are different.

In an embodiment, the stent 105 once implanted inside an artery of a person will be in direct contact with blood flow. The blood flows through the hollow interior 125 of the tube 105 and is in contact with the compressible structures 130. If the pressure in the circulatory system of the person rises above a critical or threshold pressure, which in this example is the critical buckling pressure of the compressible structures 130 that is set to match the maximum healthy blood pressure for the person, the external shells 135 of the compressible structures 130 buckle to prevent further increase in the pressure. The external shells 135 of the compressible structures 130 regain their initial shape once the pressure in the circulatory system of the person decreases, thereby resetting the stent 105.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

What is claimed is:

1. A device comprising:
a tube extending between a first end and a second end, the tube forming a rigid structure having an internal surface that defines a hollow interior area of the tube; and
one or more compressible structures seeded on and extending into the hollow interior of the tube from the internal surface, the one or more compressible structures allowing a flow path through the hollow interior area.

2. The device of claim 1, wherein each of the one or more compressible structures has an external shell defining an internal volume between the external shell and the internal surface of the tube, the external shell being deformable between a first original shape and a second deformed shape in response to a pressure increase in the hollow interior of the tube.

3. The device of claim 2, wherein the external shell is deformable based on a pressure change between a first threshold pressure and a second threshold pressure.

4. The device of claim 2, wherein the second deformed shape is one of a plurality of second deformed shapes.

5. The device of claim 4, wherein the internal volume of the second deformed shape decreases as pressure is increased in the hollow interior of the tube.

6. The device of claim 2, wherein the external shell of the one or more compressible structures has a surface that is at least partially spherical.

7. The device of claim 2, wherein the internal volume is a vacuum.

8. The device of claim 7, wherein the first threshold pressure is determined by material properties of the external shell and a ratio of the thickness to the radius of the external shell.

9. The device of claim 2, wherein a fluid is fully enclosed within the external shell.

10. The device of claim 9, wherein the fluid is air.

11. The device of claim 9, wherein the first threshold pressure is determined by material properties of the external shell, an internal pressure of the fluid within the external shell, and a ratio of the thickness to the radius of the external shell.

12. The device of claim 3, wherein the tube is in the form of a stent that is adapted for placement in a blood vessel.

13. The device of claim 12, wherein the first threshold pressure is adapted to be a maximum healthy blood pressure.

14. The device of claim 1, wherein the flow path is configured to receive a fluid.

15. The device of claim 14, wherein the fluid is a liquid.
